(19) United States
(12) Patent Application Publication
(10) Pub. No.: US 2022/0096776 A1
(43) Pub. Date: Mar. 31, 2022

(54) TUBE ADAPTER FOR NON-INVASIVE MECHANICAL VENTILATION WITH INHALATION PORT AND ORIFICE OCCLUDER

(71) Applicants: Oscar Ivan QUINTERO OSORIO, Cali (CO); Fundación Valle de Lili, Cali (CO)

(72) Inventor: Oscar Ivan QUINTERO OSORIO, Cali (CO)

(21) Appl. No.: 17/270,861

(22) PCT Filed: Aug. 21, 2019
(86) PCT No.: PCT/IB2019/057026

§ 371 Date: May 7, 2021

(30) Foreign Application Priority Data
Aug. 24, 2018 (CO) NC2018/0008861

(51) Int. Cl. A61M 16/08; A61M 16/06; A61M 16/01

(52) U.S. Cl. CPC A61M 16/0816; A61M 16/0666; A61M 16/0891; A61M 16/01; A61M 16/0683

(57) ABSTRACT

The present invention relates to an adapter for non-invasive mechanical ventilation probes with an inhalation port and orifice occluder. Specifically, the invention comprises a probe adapter with an inhalation port and orifice occluder, coupled to the oronasal mask disposed on a user or patient. According to the present invention, the probe adapter also receives a metered dose inhaler that further receives an anesthetic circuit with two enteral probes, a left enteral probe and a right enteral probe. The technical field of the invention belongs to the field of medical devices for respiratory therapy, for use in patients with assisted non-invasive mechanical ventilation and concomitant use of enteral probes with the requirement of metered dosage inhaled medicinal products.

### DESCRIPTION OF THE INVENTION FIELD OF THE INVENTION

The present invention relates to an adapter for non-invasive mechanical ventilation probes with an inhalation port and orifice occluder.

The technical field of the invention belongs to the field of medical devices for respiratory therapy, for use in patients with assisted non-invasive mechanical ventilation and concomitant use of enteral probes with the requirement of metered dosage inhaled medicinal products. The invention comprises a probe adapter with an inhalation port and orifice occluder, coupled to the oronasal mask disposed on a user or patient. According to the present invention, the probe adapter also receives a metered dose inhaler that further receives an anesthetic circuit with one or two enteral probes, a left enteral probe and a right enteral probe.

### BACKGROUND OF THE INVENTION

Non-invasive mechanical ventilation is defined as the external ventilatory support administered without need for orotracheal intubation. It is based on the supply of pressurized air and oxygen by means of a mechanical ventilator and an interface or mask in patients with respiratory impairment or at risk of same.

Non-invasive mechanical ventilation is considered to be the first choice for patients with acute respiratory insufficiency secondary to the exacerbation of chronic obstructive pulmonary disease, with pulmonary edema or a cardiac origin or for the immunosuppressed. Its use has been reported to facilitate extubation, prevent reintubation and provide comfort in end-of-life situations.

The success of non-invasive mechanical ventilation is based on a careful selection of patients, ventilator type, ventilation mode and interface. However, it is generally not recommended for managing severe acute respiratory distress syndrome, and its benefits could be lost if applied late as a rescue treatment. There are substantial reports of failure of non-invasive mechanical ventilation with frequencies from 10 to 40%. Some of these factors are patient-ventilator asynchronies, discomfort, eye irritation generated by air leakage between the interface and the patient's face, as well as skin lesions related to fitting and prolonged use of the interface, where a reported incidence of skin necrosis or abrasion have been reported of 7% to 100% after 48 hours of non-invasive mechanical ventilation, depending on the time of use of the mask.

As a matter of course, patients undergoing therapy with non-invasive mechanical ventilation concomitantly use nasogastric and/or nasojejunal probes to manage nutritional requirements, administer medication, and for gastric control, among other reasons. However, these invasive procedures increase leaks due to the presence of probes between the interface and the patient's face, leading to increased inefficient ventilation, skin lesions, and asynchrony between the patient and the ventilator.

These complications have been reduced with the device “Adapter for Non-Invasive Mechanical Ventilation Probe”, patented in the Republic of Colombia with certificate 1165 and number C013048202 by the same inventor, the international application of which is WO2014188242 A1, whereby it is possible to connect one or two enteral probes during non-invasive mechanical ventilation therapy, allowing to improve the intervention, enhance comfort levels and reduce leakage.

However, it has been found that there is a group of patients with non-invasive mechanical ventilation therapy and enteral probes that are also diagnosed with obstructive pulmonary disease who require as rescue, maintenance, or bronchospasm crisis measures the administration of medication using a metered dose inhaler (MDI).

A metered dose inhaler is a device comprising a pressurized cartridge and an external casing on which the cartridge valve is placed. Inside it the drug is dissolved or in suspension in the propellant. When exposed to atmospheric pressure it will change from liquid into gas, producing an aerosol. During spontaneous respiration, metered dose inhalers are used with devices that favor lower losses of the medication, known as inhalation chambers or spacer chambers, which can be used with a nozzle or with a mask. However, when using non-invasive mechanical ventilation therapy, the metered dose inhaler is coupled to the respiratory circuit using inhalation devices such as: AeroChamber® MV, Airlife™ Dual Spray Minispacer, Airlife™ MediSpacer Chamber, Hudson RCI® MDI Adaptor, DHD ACE™ Reservoir, Monoghan AeroVent™ Reservoir. These devices allow administering the medication, but a large amount of same is lost due to the leakage generated by the enteral probes between the mask and the patient's face. The use of the adapter for non-invasive mechanical ventilation probes allows coupling enteral probes to the interface, preventing leakage efficiently. However, the distance between the point of administration of the medication and the patient's airway reduces the reception of the medication, such that the application of the metered dose inhaler in this population can still be improved significantly.

Moreover, another situation that arises when patients have two probes is that if during their management one of the probes is removed, the adapter for non-invasive mechanical ventilation probes must be changed to one having a single orifice, depending on the size of the probe, which increases health care costs. For this reason, the possibility has been developed of occluding the orifice through which the probe that is removed, so that the device does not have to be changed.

In the effort to continue improving these situations and provide continuity to the benefits provided by this technology, the inventor of the adapter for non-invasive mechanical ventilation probes has developed a novel adapter device for non-invasive mechanical ventilation probes with an inhalation port and orifice occluder made from plastic material, that allows administering medication using metered dose inhalers frontally in the patient's airway and also allows occluding an orifice if one of the probes is removed, or conversely opening the orifice to allow entry of a new probe.

### OBJECTIVE OF THE INVENTION

Consequently, a first objective of the present invention is to overcome the drawbacks of the prior art. More specifically, an objective of the present invention is to create an adapter device for non-invasive mechanical ventilation probes with an inhalation port and orifice occluder.

Another important objective of the invention comprises an efficient dosing of inhalators in patients with two probes, such that it is not necessary to change the device while the dose is administered by inhalation.

An equally important objective of the present invention is to provide a device that efficiently allows passage of one or two probes with the option of occluding the orifice through which the removed probe passes, without having to change the device.

The novel features considered to form the basis of the invention are set out in particular in the accompanying claims, and the additional advantages thereof will be better understood from the detailed description provided below of the preferred embodiments and with reference to the accompanying drawings.

### BRIEF DESCRIPTION OF THE DRAWINGS

To further clarify the invention and the advantages thereof compared to the prior art, several possible embodiments of the application of these principles are described below with the aid of the accompanying drawings, which are provided for purposes of illustration only and in a non-limiting sense.

FIG. 1. Shows a view of the adapter with an inhaler port and orifice occluder in use according to the present invention.

FIG. 2. Shows an isometric view of the adapter with an inhaler port and orifice occluder according to the present invention.

FIG. 3. Shows a front view of the adapter with an inhaler port and orifice occluder according to the present invention.

FIG. 4. Shows a rear view of the adapter with an inhaler port and orifice occluder according to the present invention.

FIG. 5. Shows a rear left view of the adapter with an inhaler port and orifice occluder according to the present invention.

FIG. 6. Shows a top view of the adapter with an inhaler port and orifice occluder according to the present invention.

FIG. 7. Shows a first isometric view of the open adapter with an inhaler port and orifice occluder according to the present invention.

FIG. 8. Shows a second isometric view of the open adapter with an inhaler port and orifice occluder according to the present invention.

FIG. 9. Shows a front view of the adapter with an inhaler port and orifice occluder according to the present invention.

FIG. 10. Shows a front and side view of the first configuration of the orifice ring according to the present invention.

FIG. 10a. Shows a front and side view of the second configuration of the orifice ring according to the present invention.

FIG. 10b. Shows a front and side view of the configuration of the orifice occluder according to the present invention.

FIG. 11. Shows a front view of the adapter with an inhaler port and orifice occluder including the orifice occluders according to the present invention.

FIG. 12. Shows a view of the configuration of the orifice ring and its section according to the present invention.

FIG. 12a. Shows an isometric view of the configuration of the orifice ring and the manner in which it embraces the probe according to the present invention.

FIG. 12b. Shows a side view of the configuration of the orifice ring and the manner in which it embraces the probe according to the present invention.

### DETAILED DESCRIPTION OF THE INVENTION

The present invention relates to an adapter for non-invasive mechanical ventilation probes with an inhalation port and orifice occluder.

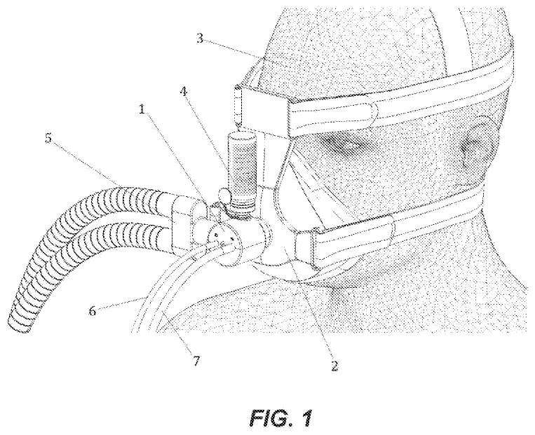

FIG. 1 shows the probe adapter 1 with an inhaler port and orifice occluder according to the present invention, coupled to the oronasal mask 2 placed on a user or patient 3.

According to the present invention, the probe adapter 1 also receives a metered dose inhaler 4 that further receives an anesthetic circuit 5 with two enteral probes, a left enteral probe 6 and a right enteral probe 7.

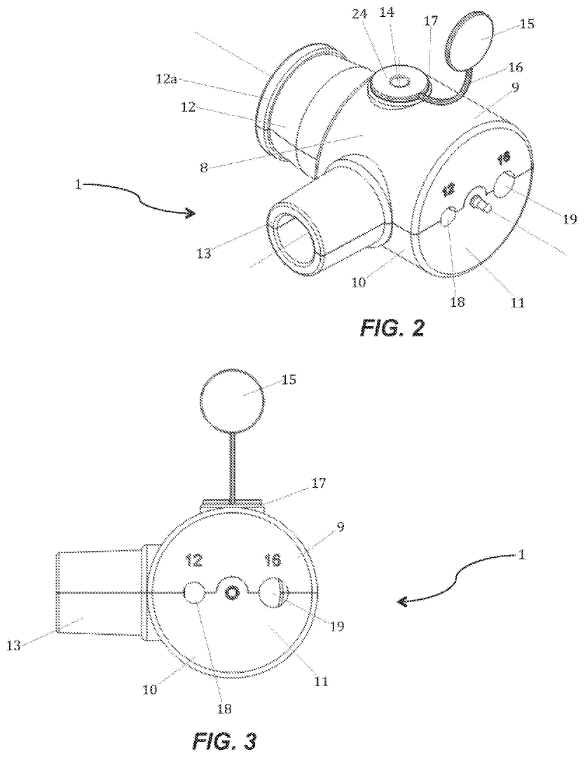

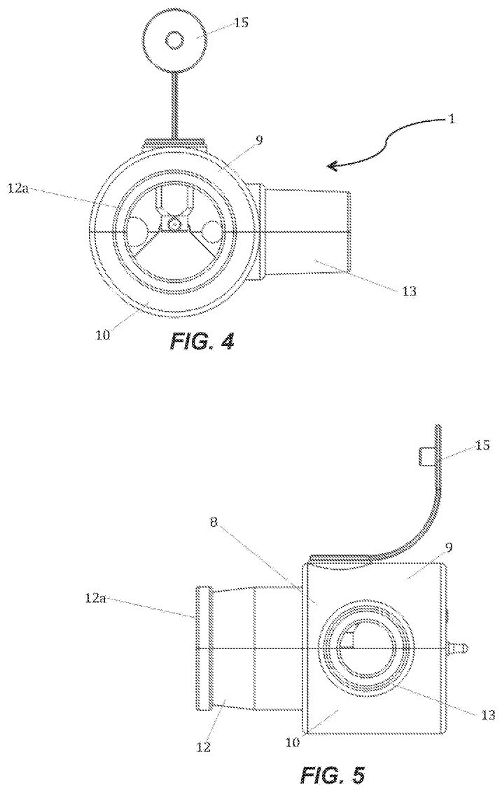

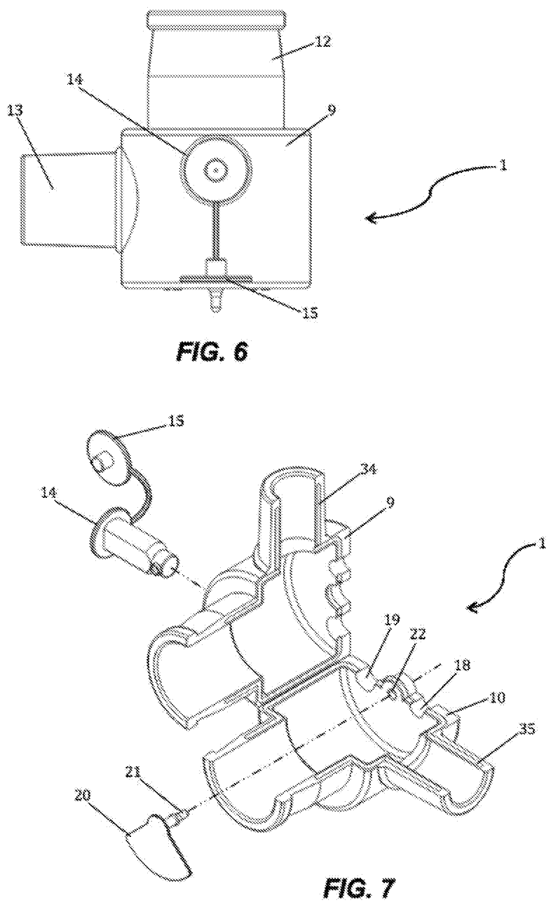

According to FIGS. 2 to 6, the adapter 1 of the invention comprises a main body 8 with a cylindrical shape whose axis is horizontal, horizontally split in half, formed from an upper casing 9 and a bottom casing 10 joined to each other. Said main body 8 ends on one extremity at a front wall 11 and at the other extremity projects a coupling tube 12 that receives the oronasal mask 2. The coupling tube 12 comprises towards its flange a retaining ring 12a that couples to the oronasal mask 2. In preferred embodiments the coupling tube 12 comprises a narrowing when projected out from the adapter, thereby improving the coupling to the oronasal mask 2.

In addition, the main body 8 of the adapter 1 comprises a lateral tube 13 that runs on the left side and receives the anesthetic circuit 5.

Above the main body 8 is a coupling port 14 that receives the metered dose inhaler 4. In a preferred embodiment of the invention, said coupling port 14 comprises a lid 15 that closes and isolate said inhaler coupling port 14.

In other embodiments the lid 15 comprises a closing pin 15a at the coupling port 14.

On the front wall 11 the invention provides the inlet orifices for the probes. For this purpose said front wall 11 comprises a first left inlet orifice 18 for the left enteral probe 6 and a second right inlet orifice 19 for the right enteral probe 7.

In the preferred embodiments the first left orifice 18 has a smaller diameter than the second right orifice 19 to allow inserting a no. 12 probe in the first left orifice 18 and a no. 16 probe in the second right orifice 19.

With reference now to FIG. 7, the inside of the adapter comprises a gate 20 arranged internally that pivots about a shaft 21 which is perpendicularly projected out of the body 8 through an orifice 22 made in the center of the front wall 11 adjacent to the first left orifice 18 and a second right orifice 19. Specifically, said gate 20 comprises a quarter-circle plate where at the center of the circumscribed circle said shaft 21 is projected perpendicularly towards the orifice 22.

In this way the gate 20 revolves about the shaft 21, occluding at the user's will the first left orifice 18 and opening the second right orifice 19 to insert a no. 16 probe in the second right orifice 19.

Alternatively, the gate 20 revolves about the shaft 21 occluding at the user's will the second right orifice 19 and opening the first left orifice 18 to insert a no. 12 probe in the first left orifice 18.

In alternative embodiments, as the gate has a quarter-circle shape, it can also open both probe orifices at the same time in an intermediate position.

In addition, as shown in FIG. 8, the coupling port 14 comprises a semi-cylindrical elongate chamber 23 with a flat side comprising a neck 24 formed by a securing ring. Said coupling port 14 is disposed above the interior of the main body 8 extending beyond a semicircular through orifice 25 with a straight side, arranged to fully receive the coupling port 14 to the neck 24 towards the interior of the main body 8.

In addition, said lid 15 comprises an extension loop 16 coupled by a ring 17 secured to said neck 24 on the outer flange of the coupling port 14.

On the end opposite the neck 24, the coupling port has a nozzle 26 oriented perpendicular to the axis of the coupling port 14. Thereby, when the inhaler is connected, the dose enters the elongate semi-cylindrical chamber 23 and is projected by the nozzle 26 sideways towards the user in the direction of the coupling tube 12 that receives the oronasal mask 2.

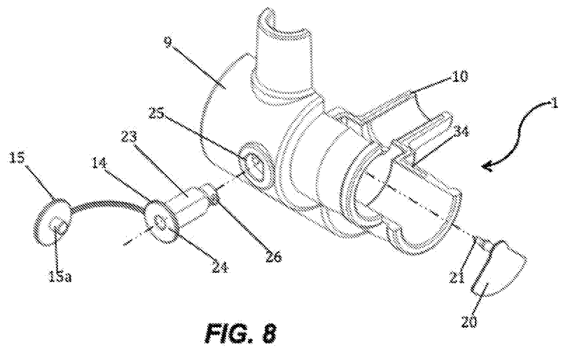

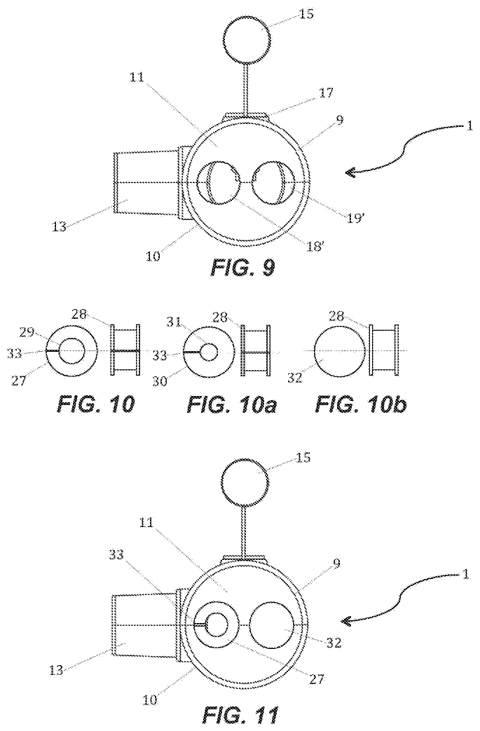

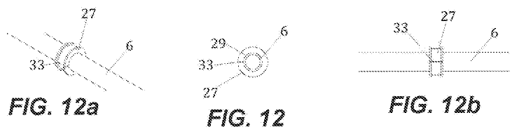

In alternative embodiments, with reference to FIG. 9 or 12b, the first left orifice 18′ can comprise a diameter equal to the second right orifice 19′, as shown in FIG. 9. In this alternative embodiment, in order to receive different probe types coupling rings with a variable inner diameter are provided. For this purpose, as shown in a top and side view of FIG. 10, a first coupling ring 27 is provided that comprises a cylindrical configuration with an extended lip 28 to provide a seal inside the orifice. The inner diameter 29 of the coupling ring 27 is arranged to receive a no. 16 probe.

These rings are made from a flexible plastic material comprising a side slit 33 for embracing the probe and then coupling to the adapter, as shown in FIGS. 12, 12a and 12b.

Alternatively, FIG. 10a defines a second coupling ring 30 where the inner diameter 31 is arranged to receive a no. 12 probe, also comprising a side slit 33.

Similarly, in embodiments where the device must not include a probe or must include a single probe, it is necessary to occlude one or both of the orifices, for which purpose an orifice occluder 32 is provided as shown in FIG. 10b, where the orifice occluder 32 is configured as a plug.

The three types of accessories for the orifice include said extended lip 28 for sealing the interior of the left orifice 18′ or right orifice 19′ where it is used as needed.

As shown in FIG. 11, as needed any of the rings can be placed inside a first orifice, occluding the second orifice at will and arranging the participation of each of the three types of accessory according to the patient's needs.

For illustration purposes, to couple the ring 27 to a probe 6, as shown in FIGS. 12, 12a and 12b, the ring 27 is opened at its slit 33 to receive the probe 6 inside it. Once they are coupled, the ring and the probe are connected in the adapter of the present invention in the orifice 18′ of FIG. 9.

In alternative embodiments, as shown in FIG. 7, the union flange between the upper casing 9 and the lower casing 10 is provided with a lip 34 that protrudes from the flange of the upper casing 9 to insert in a groove 35 provided in the flange of the lower casing 10, thereby ensuring a seal when fitting the upper casing 9 on the lower casing 10 to form the body 8 of the adapter 1.

To implement the probe adapter according to the present invention, depending on the diameter of the probe or probes of the patient, each probe must be fitted in each left orifice 18 or right orifice 19 placing them on the lower casing 10 on the semicircles formed by each orifice when the casings are opened, to close and be embraced by the upper casing 9.

Then the oronasal mask must be coupled to the coupling tube 12, towards its central orifice, secured by the retaining ring 12a. In addition, the side tube 13 is embraced by the circuit of the ventilator 5, which also acts as a closing mechanism for the adapter.

To administer the inhaled medication, it is necessary to lift the lid 15 that is joined by loop 16 to the coupling port 14 for inhaling, which is coupled to the opening 25 thereby adjusting the metered dose inhaler 4 to the coupling port 14. Its use requires synchronizing the inspiration of the patient with the administration of the medication, optimizing delivery by placing it directly in the airway projected by the nozzle 26. The mask itself acts as a reservoir, reducing risk of losses without requiring the disconnection of non-invasive mechanical ventilation therapy. Finally, the lid 15 must be coupled and adjusted to close the coupling port 14 with the closing pin 15a.

To occlude the left orifice 18 or right orifice 19, the gate 20 is turned to the left or to the right by its actuation shaft 21, so that the gate 20 occludes or opens the orifice of the probe that has been removed.

As an alternative of the invention orifice accessories are provided with an adapter design for coupling probes of greater or lesser diameter and as a mechanism for reducing the diameters. Both the first coupling ring 27 and the second coupling ring 30, as well as the occluder 32, are made from a flexible plastic material and have a slit 33 for receiving and embracing the enteral probes of any caliber, and then coupling in the left orifice 18′ or right orifice 19′ according to this alternative embodiment shown in FIGS. 9 to 12b.

The adapter described herein and illustrated in the figures represents an improvement over the technique of non-invasive mechanical ventilation in patients with enteral probes, in the function of improving delivery of the medication with a metered dose inhaler, without requiring the disconnection of the non-invasive mechanical ventilation therapy and allowing continuity thereof if one of the enteral probes is removed.

Only some preferred embodiments of the invention have been illustrated by way of example. In this regard, it can be seen that the construction of the adapter for non-invasive mechanical ventilation probes with an inhalation port and orifice occluder, as well as the rotation arrangements, can be selected from among a plurality of alternatives without departing from the scope of the invention according to the following claims.

1. Probe adapter characterized in that it comprises:
A main cylindrical body (8) with its axis in a horizontal position divided into halves formed from an upper casing (9) and a lower casing (10), joined to each other, where said main body (8) ends on one extremity at a front wall (11) and on the other extremity projects a coupling tube (12) that includes in its flange a retaining ring (12a), where in addition the main body (8) comprises a side tube (13) and where the front wall (11) comprises a first left orifice (18) and a second right orifice (19);
wherein the main body (8) has a coupling port (14) that comprises an elongate semi-cylindrical chamber (23) with a flat side and that comprises a neck (24) formed by a retaining ring, wherein said coupling port (14) is disposed above and inside the main body (8) extending beyond a semicircular through orifice (25) with a straight side, wherein the end opposite the neck (24), the coupling port (14) has a nozzle (26) with a projection perpendicular to the axis of the coupling port (14); and,
a gate (20) disposed inside the main body (8) that pivots about an actuation shaft (21) that protrudes out of said body (8), passing through an orifice (22) made in the center of the front wall (11), adjacent to the first left orifice (18) and to the second right orifice (19).

2. Probe adapter according to claim 1 characterized in that the coupling port (14) comprises a lid (15) that comprises an extension loop (16) coupled by a ring (17) secured to the outer flange of the neck (24) of the coupling port (14); and wherein the lid (15) includes a closing pin (15a).

3. Probe adapter according to claim 2 characterized in that said gate (20) comprises a quarter-circle plate in which, at the center of the circumscribed circle, said actuation shaft (21) is projected perpendicularly towards the orifice (22).

4. Probe adapter according to claim 3 characterized in that the joining flange between the upper casing (9) and the lower casing (10) has a lip (33) that protrudes from the flange of the upper casing (9) that is inserted in a groove (34) made in the flange of the lower casing (10).

5. Probe adapter according to claim 4 characterized in that the first left orifice (18) has a different diameter than that of the second right orifice (19).

6. Probe adapter according to claim 4 characterized in that the first left orifice (18′) has the same diameter as that of the second right orifice (19′).

7. Probe adapter according to claim 6 characterized in that it comprises a first cylindrical coupling ring (27) with an extended lip (28) and a second cylindrical coupling ring (30) with an extended lip (28), wherein the first and second coupling rings (27, 30) have different inner diameters and have a slit (33).

8. Probe adapter according to claim 7 characterized in that it comprises an orifice occluder (32) that forms a plug.

9. Probe adapter according to claim 8 characterized in that the first cylindrical coupling ring (27) and the second cylindrical coupling ring (30) are respectively disposed in the first left orifice (18′) and the second right orifice (19′).

10. Probe adapter according to claim 8 characterized in that the first cylindrical coupling ring (27) or the second cylindrical coupling ring (30) are disposed in combination with the orifice occluder (32) in the first left orifice (18′) and in the second right orifice (19′) respectively.
